(19) United States
(12) Patent Application Publication
(10) Pub. No.: US 2022/0348529 A1
(43) Pub. Date: Nov. 3, 2022

(54) CATALYST FOR SYNTHESIZING DIMETHYL ETHER FROM SYNTHETIC GAS, METHOD FOR MANUFACTURING THE SAME, AND METHOD FOR SYNTHESIZING DIMETHYL ETHER USING THE SAME

(71) Applicants: HYUNDAI MOTOR COMPANY, Seoul (KR); Kia Corporation, Seoul (KR); Research & Business Foundation SUNGKYUNKWAN UNIVERSITY, Suwon-si (KR)

(72) Inventors: Chae Hwan HONG, Seoul (KR); Jin Woo CHOUNG, Suwon-si (KR); Young Gul HUR, Uiwang-si (KR); Jong Wook BAE, Suwon-si (KR); Hyun Seung JUNG, Suwon-si (KR); Xu WANG, Suwon-si (KR)

(21) Appl. No.: 17/410,322

(22) Filed: Aug. 24, 2021

(30) Foreign Application Priority Data
Apr. 29, 2021 (KR) 10-2021-0055653

(51) Int. Cl. C07C 41/01; B01J 29/68; B01J 37/30; B01J 37/10; B01J 37/08; B01J 37/03; B01J 35/10

(52) U.S. Cl. CPC C07C 41/01; B01J 29/68; B01J 37/30; B01J 37/10; B01J 37/082; B01J 37/035; B01J 35/1061; B01J 35/1066; B01J 2229/18

(57) ABSTRACT

A method of preparing a catalyst for synthesizing dimethyl ether from synthetic gas includes preparing a mesoporous ferrierite zeolite (FER), and co-precipitating a precursor of a mesoporous ferrierite zeolite and a Cu—Zn—Al-based oxide (CZA) to obtain a hybrid CZA/mesoFER catalyst.

### CROSS-REFERENCE TO RELATED APPLICATION

This application claims priority to and the benefit of Korean Patent Application No. 10-2021-0055653 filed in the Korean Intellectual Property Office on Apr. 29, 2021, the entire contents of which are incorporated herein by reference.

### TECHNICAL FIELD

The present disclosure relates to a catalyst for synthesizing dimethyl ether from synthetic gas composed of carbon monoxide (CO), carbon dioxide (CO₂), and hydrogen (H₂), a method for manufacturing the same, and a method for synthesizing dimethyl ether using the same.

### BACKGROUND

As concentrations of harmful substances such as CO₂, CO, CH₄, NOR, and the like in the atmosphere increase due to rapid development of chemical industries, methods of utilize these harmful substances are being actively studied by various metal catalysts.

In this process, noble metals having high reactivity may be used as the metal catalysts but are not economical. On the contrary, relatively inexpensive CZA catalysts consisting of copper, zinc, and alumina may successfully convert carbon monoxide (CO) and also efficiently synthesize methanol variously used through the chemical industries.

Furthermore, since the methanol is converted into dimethyl ether (DME) in an acid catalyst, CZA and the acid catalyst may be synthesized into a hybrid catalyst, and this reaction may be represented by Reaction Schemes 1 and 2.

CO+2H₂→CH₃OH [Reaction Scheme 1]

2CH₃OH ↔CH₃OCH₃+H₂O [Reaction Scheme 2]

On the other hand, after the conversion reaction of carbon monoxide to dimethyl ether, methyl acetate (MA) may be synthesized from dimethyl ether through a carbonylation reaction of dimethyl ether, and ethanol may be synthesized through a hydrogenation reaction of the methyl acetate. This reaction may be represented by Reaction Schemes 3 to 5.

2CO+4H₂→CH₃OCH₃+H₂O [Reaction Scheme 3]

CH₃OCH₃+CO→CH₃COOCH₃ [Reaction Scheme 4]

CH₃COOCH₃+H₂→CH₃OH+C₂H₅OH [Reaction Scheme 5]

The above reactions are very eco-friendly and economical because it is possible to selectively synthesize ethanol as well as methanol through the conversion of carbon monoxide. In addition, since DME and MA synthesized during the reaction are each used as fuel or intermediates of various pharmaceutical chemicals, carbon monoxide may be more effectively recycled by controlling these processes.

### SUMMARY

The present disclosure provides a catalyst capable of synthesizing dimethyl ether from synthetic gas, which has excellent catalytic activity, increases reactivity due to active mass transfer, and has excellent conversion rate of carbon monoxide (CO) and selectivity of dimethyl ether (DME).

The present disclosure provides a method for preparing the catalyst.

The present disclosure provides a method for preparing dimethyl ether (DME) from synthetic gas using the catalyst.

According to an embodiment, a method for forming a catalyst for synthesizing dimethyl ether from synthetic gas includes preparing a catalyst includes preparing a mesoporous ferrierite zeolite (FER), and co-precipitating a precursor of a mesoporous ferrierite zeolite and a Cu—Zn—Al-based oxide (CZA) to obtain a hybrid CZA/mesoFER catalyst.

The preparing of the mesoporous ferrierite zeolite may include preparing ferrierite, silicon-leaching the ferrierite, and performing hydrothermal synthesis of the precursor mixed solution.

The preparing of ferrierite may include adding a silica source, an alumina source, and a ferrierite seed to a basic aqueous solution to prepare a precursor mixed solution, and synthesizing ferrierite by hydrothermal synthesis of the precursor mixed solution.

The ferrierite seed may be added in an amount of about 2 wt % to about 30 wt % based on the total weight of the prepared ferrierite.

The hydrothermal synthesis of the precursor mixed solution may be performed at about 120° C. to about 180° C. for about 96 hours to about 168 hours.

The silicon-leaching of the ferrierite may be performed by adding an organic template material and ferrierite to a basic aqueous solution and stirring at about 10° C. to about 80° C. for about 1 hour to about 15 hours.

The organic template material may be a linear organic compound having 15 to 30 carbons and at least one nitrogen.

The organic template material may include cetrimonium bromide (CTAB), sodium dodecyl sulfate, ammonium lauryl sulfate, or a combination thereof.

The organic template material may be added in an amount of about 10 to about 50 parts by weight based on 100 parts by weight of ferrierite.

The hydrothermal synthesis of silicon-leached ferrierite may be performed at about 120° C. to about 180° C. for about 48 hours to about 96 hours.

The hydrothermal synthesis of silicon-leached ferrierite may further include ion-exchanging a Na-form zeolite prepared by hydrothermal synthesis of silicon-leached ferrierite with a cation to prepare a NH₃-form zeolite.

The hydrothermal synthesis of silicon-leached ferrierite may further include calcining the ion-exchanged zeolite at about 450° C. to about 650° C. for about 3 hours to about 6 hours to convert the ion-exchanged zeolite into H-from zeolite.

The co-precipitating may include preparing a first solution including mesoporous ferrierite zeolite, preparing a second solution including a copper precursor, a zinc precursor, and an aluminum precursor, and preparing a third solution including a basic precipitating agent, and adding the second solution and the third solution to the first solution to perform co-precipitating.

A mole ratio of Cu:Zn:Al in the second solution may be (about 10 to about 5):(about 5 to about 1):1.

The copper precursor may include an acetate, a hydroxide, a nitrate, or a combination thereof of copper, the zinc precursor may include an acetate, a hydroxide, a nitrate, or a combination thereof of zinc, and the aluminum precursor may include an acetate, a hydroxide, a nitrate, or a combination thereof, of aluminum.

The basic precipitating agent may include sodium carbonate, potassium carbonate, ammonium carbonate, sodium hydrogen carbonate, or a combination thereof.

In the co-precipitating process, the second solution and the third solution may be dropped dropwise to the first solution to co-precipitate the precursor of the mesoporous ferrierite zeolite and the Cu—Zn—Al-based oxide (CZA).

The co-precipitating may be performed at a temperature of about 65° C. to about 75° C. and a pH of less than or equal to about 7.

The co-precipitating may further include growing a crystal of the prepared precipitate for about 1 hour to about 2 hours.

The co-precipitating may further include calcining the prepared precipitate at about 200° C. to about 600° C. for about 2 hours to about 6 hours.

According to another embodiment, a hybrid CZA/mesoFER catalyst includes a mesoporous ferrierite zeolite and a Cu—Zn—Al-based oxide supported on the mesoporous ferrierite zeolite.

The Cu—Zn—Al-based oxide may include about 40 wt % to about 60 wt % of CuO, about 35 wt % to about 45 wt % of ZnO, and about 5 wt % to about 15 wt % of Al₂O₃ based on the total weight of the Cu—Zn—Al-based oxide.

The hybrid CZA/mesoFER catalyst may include about 0.1 part by weight to about 5 parts by weight of the Cu—Zn—Al-based oxide based on 1 part by weight of the mesoporous ferrierite zeolite.

A Si/Al ratio of the mesoporous ferrierite zeolite may be about 5 to about 30.

The mesoporous ferrierite zeolite may have mesopores having a size of about 10 nm to about 70 nm in an amount of about 80 volume % to about 30 volume %.

According to another embodiment, a method for preparing dimethyl ether includes selectively preparing dimethyl ether through a conversion reaction of synthetic gas using the hybrid CZA/mesoFER catalyst.

In the method for preparing dimethyl ether, the synthetic gas may include hydrogen (H₂) and carbon monoxide (CO) in a mole ratio of about 1:2.5 to about 1:7.5, and may include about 8 mol % to about 30 mol % of carbon monoxide based on the total amount of the synthetic gas.

The catalyst of the present disclosure has excellent catalytic activity, increased reactivity due to active mass transfer, and excellent carbon monoxide (CO) conversion and selectivity of dimethyl ether (DME). Therefore, the catalyst may capture inevitably generated harmful gas to make it into synthetic gas, and then uses it to synthesize dimethyl ether and methyl acetate (MA), which are used as fuels, ultimately converting harmful substances easily while selectively synthesizing useful substances, which may lay foundation for a continuous chemical process in which useful substances can be easily synthesized, and at the same time, may solve environmental problems and create huge economic benefits.

### BRIEF DESCRIPTION OF THE DRAWINGS

FIG. 1 is a graph showing the XRD measurement results of the hybrid CZA/mesoFER catalyst in Experiment 3.

FIG. 2 is a graph showing the N₂-sorption measurement result of the hybrid CZA/mesoFER catalyst in Experiment 3.

FIG. 3 is a photograph showing the TEM measurement result of the hybrid CZA/mesoFER catalyst in Experiment 3.

FIG. 4 is a graph showing the measurement result of N₂O-chemisorption of the hybrid CZA/mesoFER catalyst in Experiment 4.

FIGS. 5A-5C show a graph showing the XPS measurement results of the hybrid CZA/mesoFER catalyst in Experiment 4.

### DETAILED DESCRIPTION OF THE EMBODIMENTS

The advantages and features of the present disclosure and the methods for accomplishing the same will be apparent from the embodiments described hereinafter with reference to the accompanying drawings. However, the embodiments should not be construed as being limited to the embodiments set forth herein. Unless otherwise defined, all terms (including technical and scientific terms) used herein have the same meaning as commonly understood by one of ordinary skill in the art. In addition, terms defined in a commonly used dictionary are not to be ideally or excessively interpreted unless explicitly defined.

In addition, unless explicitly described to the contrary, the word “comprise,” and variations such as “comprises” or “comprising,” will be understood to imply the inclusion of stated elements but not the exclusion of any other elements.

Further, the singular includes the plural unless mentioned otherwise.

A method of preparing a catalyst according to an embodiment includes preparing a mesoporous ferrierite zeolite (FER), and co-precipitating a precursor of a mesoporous ferrierite zeolite and a Cu—Zn—Al-based oxide (CZA) to obtain a hybrid CZA/mesoFER catalyst. The catalyst may be used as a catalyst for synthesizing dimethyl ether from synthetic gas.

For example, the preparing of the mesoporous ferrierite zeolite may include preparing ferrierite, silicon-leaching the ferrierite, and performing hydrothermal synthesis of the precursor mixed solution.

As the ferrierite, commercially available ferrierite may be used, or ferrierite synthesized using commercially available ferrierite as a seed may be used.

For example, when the ferrierite is synthesized, the preparing of ferrierite may include adding a silica source, an alumina source, and a ferrierite seed to a basic aqueous solution to prepare a precursor mixed solution, and synthesizing ferrierite by hydrothermal synthesis of the precursor mixed solution.

The basic aqueous solution may be an aqueous alkali hydroxide solution including sodium hydroxide, potassium hydroxide, calcium hydroxide, magnesium hydroxide, or a combination thereof.

The silica source may include silica sol, silica gel, silica hydrogel, silica hydroxide, fumed silica, precipitated silica, sodium silicate, tetraalkylorthosilicate, or combinations thereof.

The aluminum source may include sodium aluminate (NaAlO₂), AlCl₃, Al₂(SO₄)₃, aluminum hydroxide (Al(OH)₃), kaolin, clay, or a combination thereof.

The silica source and the aluminum source may be added in a mole ratio of about 5:1 to about 30:1, for example, about 10:1. When the mole ratio of the silica/aluminum source is less than about 5, the amount of acid sites serving as the reaction point may be too small, and the reactivity may be severely reduced. When the mole ratio of the silica/aluminum source is greater than about 30, aluminum is excessively embedded in the ferrierite structure, the strength of the acid spots may be weakened or the ferrierite characteristic crystal itself may not be formed. In one example, being “about” a value may mean that being exactly the value or being bigger or smaller than the value within a tolerance, an error, or a margin due to measurement or process variation, which is recognizable by one of ordinary skill in the art.

As the ferrierite seed, commercial ferrierite may be used, and the ferrierite seed may be added in an amount of about 2 wt % to about 30 wt %, for example, about 2 wt % to about 25 wt %, or about 7 wt % to about 24 wt % based on the total weight of the final prepared ferrierite. When the amount of the ferrierite seed is less than about 2 wt %, there may be no significant difference in physical properties from that of ferrierite used as an existing seed. When the amount of the seed exceeds about 30 wt %, the concentration of aluminum defect points may increase, resulting in reduction of catalyst performance and acceleration of deactivation.

Optionally, a structural derivative material that plays a key role in the formation of a ferrierite-specific framework structure and an organic template material that induces special physical properties may be further added to the precursor mixed solution.

The organic template material may be a linear organic compound containing 15 to 30 carbons and at least one nitrogen.

For example, the organic template material may include 10 to 30 carbons, 10 or more carbons form a chain bond, and may be a material including an ionic moiety. For example, the organic template material may include cetrimonium bromide (CTAB), sodium docecyl sulfate, ammonium lauryl sulfate, or a combination thereof.

The structural derivative material may be a nitrogen-containing heterocyclic compound including pyrrolidine, piperidine, or a combination thereof.

The structural derivative may be added in an amount of about 0.2 to about 2.0 parts by mole, for example, about 0.8 to about 1.0 part by mole based on 1 part by mole of the silica source. When the amount of the structural derivative is less than about 0.2 parts by mole, the ferrierite structure itself may not be formed, and when it exceeds about 2.0 parts by mole, the amount of acid sites generated after synthesis may decrease and thus catalyst reactivity may also decrease.

The organic template material and the structural derivative material may be added in a mole ratio of about 0.01:1 to about 0.5:1. If the amount of the organic template material is too large, the crystal structure of ferrierite itself is damaged, and synthesis may be difficult.

The hydrothermal synthesis of the precursor mixed solution may be performed at about 120° C. to about 180° C. for about 96 hours to about 168 hours. If the hydrothermal synthesis temperature is less than about 120° C., the crystallinity of the synthesized ferrierite may decrease, and if it exceeds about 180° C., the particle size of the synthesized catalyst may increase and mesopores may not be formed. If the hydrothermal synthesis time is less than about 96 hours, the crystallinity of the synthesized ferrierite may decrease, and if it exceeds about 168 hours, the crystal size of the synthesized ferrierite may become too large and mesopores may not be formed.

Silicon-leaching of ferrierite may be performed by adding an organic template material and ferrierite to a basic aqueous solution and stirring the resultant.

As the ferrierite, commercial ferrierite or seed-synthesized ferrierite may be used, and the ferrierite may be various a form including cations such as Na-form, NH₃-form, and H-form without limitation.

During the silicon-leaching, an organic template material, which plays a key role in forming the ferrierite-specific framework, may be added to the basic aqueous solution.

As the organic template material, a chain-type carbon material including about 10 to 30 carbons may be used. For example, the organic template material may include cetrimonium bromide (CTAB), sodium docecyl sulfate, ammonium lauryl sulfate, or a combination thereof.

During silicon-leaching, the organic template material may be added in an amount of about 10 to about 50 parts by weight, for example, about 20 to about 40 parts by weight based on 100 parts by weight of ferrierite. If the amount of the organic template material is less than about 10 parts by weight, silicon-leaching may not be performed, and if it exceeds about 50 parts by weight, the silicon may be excessively leached and the ferrierite structure itself may be damaged.

The silicon-leaching may be performed at about 10° C. to about 80° C. for about 1 hour to about 15 hours, for example, at about 25° C. to about 70° C. or room temperature for about 3 hours to about 12 hours. When the temperature of the silicon-leaching is less than about 10° C., the silicon is not leached sufficiently, so mesopores may not be properly formed. When the temperature of the silicon-leaching exceeds about 70° C., crystallinity may decrease and defect points may increase due to excessive silicon-leaching. When the silicon-leaching time is less than about 3 hours, the silicon is not leached sufficiently and mesopores may not be properly formed. When the silicon-leaching time exceeds about 12 hours, the crystallinity decreases and the defect points increase due to excessive silicon-leaching.

The silicon-leaching process introduces irregular pores by randomly creating a large number of defect points by attacking the already synthesized ferrierite structure using organic materials including long carbon chains. A large amount of carbon component may form pores by being embedded in the ferrierite framework and then controlled. In addition, the damaged defect point is made into a ferrierite structure again through a hydrothermal synthesis process, but the already formed pores are not destroyed.

Through silicon-leaching, mesopores having an appropriate size may be easily and uniformly introduced into a smooth plate-shaped ferrierite structure, thereby inducing an increase in specific surface area and co-precipitating Cu more effectively.

After re-hydrothermal synthesis of silicon-leached ferrierite, and optionally ion-exchange, H-form mesoporous ferrierite may be finally prepared.

Re-hydrothermal synthesis of silicon-leached ferrierite may be performed at about 120° C. to about 180° C. for about 48 hours to about 96 hours. If the re-hydrothermal synthesis temperature is less than about 120° C., the crystallinity of the synthesized ferrierite may decrease, and if it exceeds about 180° C., the particle size of the synthesized catalyst may increase and mesopores may not be formed. If the re-hydrothermal synthesis time is less than about 48 hours, the crystallinity of the synthesized ferrierite may decrease, and if it exceeds about 96 hours, the crystal size of the synthesized ferrierite may become too large and mesopores may not be formed.

In this case, the synthesized zeolite may be a Na-form zeolite, and an NH₃-form zeolite may be prepared by exchanging the Na-form zeolite with a cation through the ion-exchange.

As an example, the ion-exchange involves repeating 3 to 6 times the processes of dipping Na-form zeolite in an aqueous solution of ammonium nitrate (NH₄NO₃), and stirring the resultant at about 60° C. to about 80° C. for 3 hours or more to be exchanged into a NH₄⁺ ion form and thus to prepare NH₃-form zeolite.

Additionally, the method may further include removing impurities, structural derivative residues, and organic template residues included in the synthesized zeolite through washing the ion-exchanged zeolite with distilled water, drying at a high temperature, or calcining at a high temperature. For example, high-temperature calcination may convert the ion-exchanged zeolite into H-from zeolite by calcining the ion-exchanged zeolite at about 450° C. to about 650° C. for about 3 hours to about 6 hours. If the calcination reaction temperature is less than 450° C., the removal of ammonium ions may not be sufficient, so OH bonds (Brönsted acid sites) may not be sufficiently generated and if it exceeds about 650° C., the ferrierite structure itself may collapse. If the time is less than about 3 hours, the removal of ammonium ions may not be sufficient, so that the OH bond (Brönsted acid site) may not be sufficiently generated, and if it exceeds about 6 hours, the ferrierite structure itself may collapse.

The co-precipitating may include preparing a first solution including mesoporous ferrierite zeolite, preparing a second solution including a copper precursor, a zinc precursor, and an aluminum precursor, preparing a third solution including a basic precipitating agent, and adding the second solution and the third solution to the first solution to co-precipitate them.

For example, after dispersing the prepared mesoporous ferrierite zeolite in an aqueous solution, the mixed solution of the metal precursor including the copper precursor, the zinc precursor, and the aluminum precursor and the basic aqueous solution are simultaneously dropped and stirred in the aqueous zeolite solution, followed by stirring the resultant to prepare a hybrid CZA/mesoFER catalyst.

The first solution may be a suspended aqueous solution prepared by mixing the prepared nanosheet ferrite zeolite with an aqueous solution.

The metal precursor of the copper precursor, the zinc precursor, and the aluminum precursor in the second solution may include an acetate, a hydroxide, a nitrate, or a combination thereof, as a precursor of each metal. For example, the copper precursor may include an acetate, a hydroxide, a nitrate, or a combination thereof of copper, the zinc precursor may include an acetate, a hydroxide, a nitrate, or a combination thereof of zinc, and the aluminum precursor may include an acetate, a hydroxide, a nitrate, or a combination thereof of aluminum.

The mole ratio of Cu:Zn:Al in the second solution may be from (about 10 to about 5):(about 5 to about 1):1, for example, from (about 8 to about 6):(about 4 to about 2):1, or about 7:3:1. If the mole ratio of copper (Cu) is less than about 5, the reactivity may decrease due to insufficient formation of Cu that is the reaction point and if the mole ratio of copper (Cu) exceeds about 10, the reactivity may decrease or deactivation may be rapid due to severe aggregation of Cu. If the mole ratio of zinc (Zn) is less than about 1, hydrophobicity of the catalyst is weak, side reactions may proceed by water generated during the reaction, and the reactivity may be reduced, and if the mole ratio of zinc (Zn) exceeds about 5, the stability of Cu, the reaction point, may be weakened. If the mole ratio of aluminum (Al) is too small, the selectivity of the final product, dimethyl ether, may decrease, and if it is too large, the stability of Cu, the reaction point, may be weakened.

In the third solution, the basic precipitating agent may include sodium carbonate, potassium carbonate, ammonium carbonate, sodium hydrogen carbonate, or a combination thereof.

When the first solution to the third solution is prepared, the second solution and the third solution may be added dropwise to the first solution to be co-precipitated. The co-precipitating may be performed at a temperature of about 65° C. to about 75° C. and a pH of about 7 or less. By introducing the third solution including the basic precipitating agent, the pH of the solution in which the precursor solutions are dissolved may be adjusted.

After the second solution including the metal precursor is all consumed, the process of growing the crystal for about 1 to about 2 hours may be further included. In addition, the method may further include a process of optionally washing, drying, and calcining the hybrid CZA/mesoFER catalyst after co-precipitating.

The drying may be performed by heating the precipitate at a temperature of about 100° C. or higher, for example, about 100° C. to about 150° C. for one or more days, and the calcining may be performed by heat treatment at about 200° C. to about 600° C. for about 2 hours to about 6 hours. If the calcining temperature is less than about 200° C., a portion of the metal precursor remains on the surface and the production of by-products may increase. When the temperature exceeds about 600° C., the surface acidity of the solid acid catalyst is changed according to the change in the oxidation state of the metal oxide, so that the production of by-products such as CO₂ may increase.

A hybrid CZA/mesoFER catalyst according to another embodiment includes a mesoporous ferrierite zeolite and a Cu—Zn—Al-based oxide supported on the mesoporous ferrierite zeolite.

The hybrid CZA/mesoFER catalyst disperses a large amount of Cu more widely compared to commercial FER due to the large specific surface area of the mesoporous ferrierite zeolite, so that Cu, the reaction point in the dimethyl ether conversion reaction of synthetic gas, is introduced more evenly and stably. That is, the hybrid CZA/mesoFER catalyst has a high degree of Cu dispersibility, is easy to reduce, and has a larger specific surface area of Cu metal, so it is possible to improve the DME production process through the conversion reaction of CO.

The Cu—Zn—Al-based oxide may include about 40 wt % to about 60 wt % of CuO, about 35 wt % to about 45 wt % of ZnO, and about 5 wt % to about 15 wt % of Al₂O₃ based on the total weight of the Cu—Zn—Al-based oxide. If the amount of CuO is less than about 40 wt %, a yield reduction phenomenon occurs due to a decrease in the active point for methanol synthesis, and when it exceeds about 60 wt %, it is difficult to form an appropriate catalyst structure with other metals, so that the reactivity may be reduced. If the amount of ZnO is less than about 35 wt %, an appropriate porous material with CuO and Al₂O₃ may be prevented from being formed, and if it exceeds about 45 wt %, the reaction rate of methanol synthesis due to reduction of CuO, an active ingredient, may be reduced. If the amount of Al₂O₃ is less than about 5 wt %, it may be difficult to form a structure favorable to the activity of the Cu—Zn—Al-based oxide, and if it exceeds about 15 wt %, the reactivity may be reduced due to reduction of the active point for methanol synthesis.

The hybrid CZA/mesoFER catalyst may include about 0.1 part by weight to about 5 parts by weight, for example, about 0.5 parts by weight to about 4 parts by weight of the Cu—Zn—Al-based oxide based on 1 part by weight of the mesoporous ferrierite zeolite. If the amount of the Cu—Zn—Al-based oxide is less than about 0.1 parts by weight, the activity for the methanol synthesis reaction may decrease and the yield of the entire process may decrease due to an increase in the conversion rate to CO₂, and if it exceeds about 5 parts by weight, the conversion rate to dimethyl ether may decrease due to the decrease of the active point of the solid acid catalyst.

A Si/Al mole ratio of the mesoporous ferrierite zeolite may be about 5 to about 30. If the Si/AI mole ratio of the mesoporous ferrierite zeolite is less than about 5, the amount of acid sites serving as a reaction point may be too small to severely decrease reactivity, and if it exceeds about 30, aluminum may be excessively embedded in the ferrierite structure, and on the contrary, the strength of the acid sites may be weakened or the ferrierite characteristic crystal itself may not be formed.

The mesoporous ferrierite zeolite may include mesopores having a size of about 10 nm to about 70 nm in an amount of about 80 volume % to about 30 volume % based on the total pore volume, and may include, for example, mesopores having a size of about 30 nm to about 50 nm in about 70 volume % to about 80 volume %. If the size of the mesopores is less than about 10 nm, the mass transfer ability may not be improved, and if it exceeds about 70 nm, the crystallinity of the catalyst itself may decrease. When the volume of the mesopores is less than about 30 volume % based on the total pore volume, the mass transfer ability may not be improved, and when it exceeds about 80 volume %, the crystallinity of the catalyst itself may decrease.

The catalyst of the present disclosure has a Bronsted acid site of copper (Cu) and ferrierite (FER) as a reaction point, so that carbon monoxide (CO) reacts with hydrogen (H₂) on Cu to be converted to methanol, and methanol is converted to dimethyl ether (DME) at the Bronsted acid site of the ferrierite. Specifically, FER provides a reaction point at which methanol is converted to dimethyl ether and serves as a support in which Cu, which is a reaction point of carbon monoxide, is dispersed.

In particular, since the catalyst contains mesoporous FER, catalytic activity may be remarkably increased, and mass transfer is more active and reactivity is also increased. Specifically, by introducing mesopores into ferrierite, it is possible to improve the physical properties of ferrierite and at the same time increase the dispersibility of Cu to prevent the Cu particles from being agglomerated or easily oxidized. These effects may lead to increased CO conversion and increased DME selectivity.

Accordingly, the catalyst may be successfully converted from synthetic gas composed of carbon monoxide (CO), carbon dioxide (CO₂), and hydrogen (H₂) to dimethyl ether. In addition, dimethyl ether may be easily converted to the important chemical material, methyl acetate (MA). At this time, the reacting CO and CO₂ are representative environmental pollutants, which are generated after chemical reaction in many industrial sites such as steel mills and factories and are harmful gases that are discharged into the atmosphere. Therefore, the catalyst captures the inevitably generated harmful gas, makes it into synthetic gas, and then uses it to synthesize DME/MA used as a fuel, ultimately converting harmful substances easily and selectively synthesizing useful substances, which may lay the foundation for a continuous chemical process that exists, and at the same time solve environmental problems, and create enormous economic benefits.

The method for preparing dimethyl ether according to another embodiment may selectively prepare dimethyl ether through the conversion reaction of synthetic gas using the hybrid CZA/mesoFER catalyst.

In the method for preparing dimethyl ether, the synthetic gas may include hydrogen (H₂) and carbon monoxide (CO) in a mole ratio of about 1:2.5 to about 1:7.5, and may include about 8 mol % to about 30 mol % of carbon monoxide based on the total amount of the synthetic gas. When the synthetic gas includes carbon monoxide in a mole ratio of less than about 2.5, the final dimethyl ether productivity may decrease, and when the carbon monoxide is included in a mole ratio of more than 7.5, the carbon monoxide conversion rate may be lowered. In addition, when the synthetic gas includes carbon monoxide in a mole ratio of less than about 8 mol %, the final dimethyl ether productivity may decrease, and when the carbon monoxide is included in a mole ratio of more than 30 mol %, the carbon monoxide conversion rate may be lowered.

Hereinafter, specific embodiments of the disclosure are presented. However, the examples described below are only for specifically illustrating or explaining the disclosure, and the scope of the disclosure is not limited thereto.

### [Experiment 1: Synthesis of Hybrid CZA/mesoFER Catalyst]

1) Synthesis of Mesoporous Ferrierite

1-1) Synthesis of Precursor Ferrierite Through Seed Synthesis of Commercial Ferrierite

Commercial ferrierite (a mole ratio of Si/Al:about 10) was used as a seed and added to a basic silica solution and completely dissolved therein through stirring for a predetermined period of time. Subsequently, an alumina precursor was added thereto and then, completely stirred to perform a hydrothermal synthesis at 160° C. for 4 days. Herein, a fraction of the added commercial ferrierite seed was advantageously in a range of 5 wt % to 30 wt %. After completing the seed synthesis through the hydrothermal synthesis, the synthesis was completed through washing, drying, and calcining.

1-2) Silicon-Leaching

The seed-synthesized catalyst by using the commercial ferrierite seed in the 1-1) was dissolved in a basic aqueous solution in which CTAB was dissolved to perform silicon-leaching. Herein, after dissolving 3 g of CTAB in 200 ml of a 0.25 M NaOH aqueous solution, 6 g of the seed-synthesized ferrierite was added thereto and then, stirred at room temperature for 3 hours. After the stirring, the synthesized catalyst was washed and dried, obtaining powder-type silicon-leached ferrierite particles.

1-3) Re-Hydrothermal Synthesis

The silicon-leached catalyst prepared in the 1-2) was added to the basic silica solution of the 1-1), and an alumina source was added thereto again and then, stirred, performing the hydrothermal synthesis again. Herein, as in the 1-1), after performing the re-hydrothermal synthesis at 160° C. for 4 days, washing, drying, and calcining were performed, obtaining Na-form ferrierite re-hydrothermally synthesized after the silicon-leaching.

1-4) Ion-Exchange

The Na-form ferrierite prepared in the 1-3) was dissolved in a solution in which 1 M of an ammonium precursor was dissolved and then, stirred at 80° C. for 3 hours and subsequently, washed and dried to perform an ion-exchange. The dried catalyst was ion-exchanged again to carry out six ion-exchanges in total and then, calcined, finally obtaining H-form mesoporous ferrierite, which is called mesoFER.

2) Co-Precipitating

The prepared mesoporous ferrierite was used to co-precipitate Cu/ZnO/Al₂O₃, and a first solution, a second solution, and a third solution were prepared for this process.

The synthesized mesoporous ferrierite and commercial ferrierite respectively by 1 g were dissolved in 200 ml of distilled water, preparing a first solution including ferrierite supports.

A second solution was prepared by completely dissolving copper nitrate, zinc nitrate, and aluminum nitrate in 200 ml of distilled water to have a mole ratio of Cu:Zn:Al=7:3:1.

A third solution as a basic precipitating agent with pH 7 or higher was prepared by completely dissolving 7 g of ammonium carbonate in 200 ml of distilled water.

The second solution and the third solution were simultaneously added dropwise to the first solution at an appropriate rate, and simultaneously, the first solution was maintained at 75° C. and pH 7. The second solution was all dropped to the first solution and then, aged, while 75° C. and pH 7 were maintained for 1 hour. After the aging, precipitates therein were washed and dried and then, calcined at 350° C. for 3 hours to complete a hybrid CZA/mesoFER synthesis.

Depending on a used FER support, the synthesized products were called to be CZA/mesoFER and CZA/CFER. CZA/mseoFER and CZA/CFER were respectively synthesized by performing co-precipitation in mesoFER, which is mesoporous ferrierite, and commercial ferrierite.

### Example 1

CZA/mesoFER catalysts were prepared according to Experiment 1.

### Comparative Example 1

CZA/CFER catalysts were prepared according to Experiment 1.

[Experiment 2: Conversion Reaction Experiment of Dimethyl Ether from Synthetic Gas]

0.4 g of the synthesized hybrid CZA/mesoFER as a catalyst for a conversion reaction of synthetic gas into dimethyl ether was loaded in a ⅜ inch fixed bed reaction. Before the reaction, a mixed gas of H₂/N₂=5/95 was used for reduction at 350° C. under a normal pressure for 5 hours. After the reduction, a reaction experiment was conducted with synthetic gas including carbon monoxide and carbon dioxide (CO/CO₂/N₂/H₂=21/9/4/66 and CO/CO₂/N₂/H₂/CH₄=8/8/2/60/22), wherein a reaction pressure was 50 bars, and a space velocity was fixed at 5000 L/kg_{cat}/h. In order to verify reactivity depending on a temperature, a dimethyl ether synthesis reaction was performed at 220° C., 250° C., 270° C., 290° C. each for 10 hours and also, at a fixed temperature of 270° C. for 40 hours.

Products from the reactions were analyzed with respect to compositions through gas chromatography, and the analysis results was used to calculate a carbon monoxide conversion rate, methanol and dimethyl ether selectivity, a dimethyl ether production, etc. during the dimethyl ether synthesis form the synthetic gas. In addition, in the conversion reaction of dimethyl ether to methylacetate, a dimethyl ether conversion rate, methylacetate selectivity, a deactivation rate, etc. were calculated. The deactivation rate was defined as an average conversion rate from at a maximum point of the conversion rate to an end point thereof.

The synthesis reactions of dimethyl ether from the synthetic gas are summarized in Tables 1 to 3.

TABLE 1

| | | CO | | Inac- | DME |
|---|---|---|---|---|---|
| Gas | | conver- | DME | tivation | produc- |
| composition/ | | sion | selec- | rate | tivity |
| temperature | | rate | tivity | [%/h · | [g_{DME}/ |
| [CO/CO₂/ | | max. | max. | (m²/ | kg_{cat} · |
| H₂]/[° C.] | Catalyst | [%] | [%] | Cu)] | h] |
| 21/9/66/270 | Example 1/ | 43.2 | 96.0 | 0.025 | 711.8 |
| | CZA/ | | | | |
| | mesoFER | | | | |
| | Comparative | 25.5 | 92.7 | 0.021 | 410.3 |
| | Example 1/ | | | | |
| | CZA/CFER | | | | |

| | | | CO | | DME |
|---|---|---|---|---|---|
| | | | conver- | DME | produc- |
| Gas | | | sion | selec- | tivity |
| composition | | | rate | tivity | [g_{DME}/ |
| [CO/CO₂/ | | Temper- | max. | max. | kg_{cat} · |
| H₂] | Catalyst | ature | [%] | [%] | h] |
| 21/9/66 | Example 1/ | 220 | 9.0 | 76.1 | 155.2 |
| | CZA/ | 250 | 37.0 | 88.3 | 574.9 |
| | mesoFER | 270 | 52.1 | 89.6 | 810.3 |
| | | 290 | 53.8 | 89.2 | 838.4 |
| | Comparative | 220 | 2.2 | 70.8 | 61.3 |
| | Example 1/ | 250 | 10.5 | 80.5 | 191.6 |
| | CZA/CFER | 270 | 22.5 | 84.6 | 370.7 |
| | | 290 | 39.9 | 87.5 | 638.2 |

TABLE 3

| | | | CO | | DME |
|---|---|---|---|---|---|
| | | | conver- | DME | produc- |
| Gas | | | sion | selec- | tivity |
| composition | | | rate | tivity | [g_{DME}/ |
| [CO/CO₂/ | | Temper- | max. | max. | kg_{cat} · |
| H₂] | Catalyst | ature | [%] | [%] | h] |
| 8/8/60 | Example 1/ | 220 | 4.4 | 64.8 | 39.6 |
| | CZA/ | 250 | 19.3 | 82.3 | 133.5 |
| | mesoFER | 270 | 34.3 | 86.7 | 238.2 |
| | | 290 | 40.6 | 87.2 | 281.7 |

Referring to Tables 1 to 3, Example 1 which was co-precipitated in mesoporous ferrierite exhibited improved catalytic activity and high DME productivity, regardless of the change in reaction conditions.

### [Experiment 3: Physical/Structural Analysis of Hybrid CZA/mesoFER Catalyst]

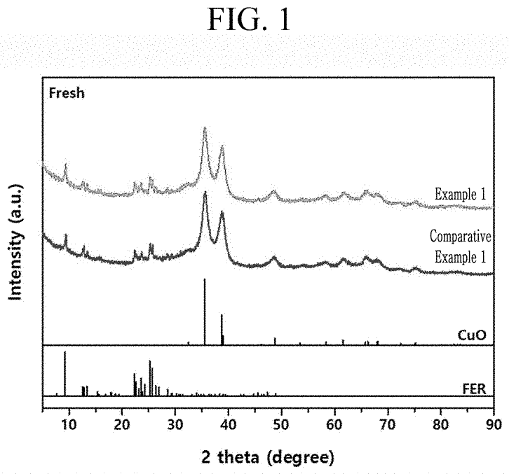

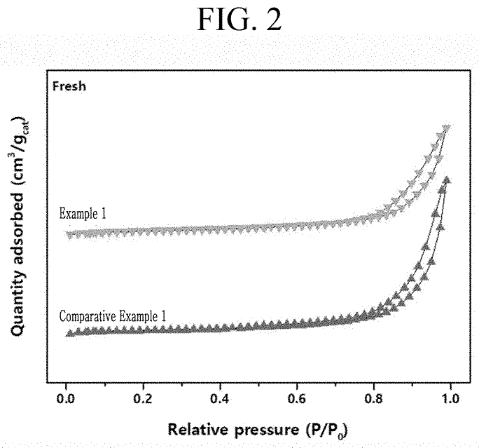

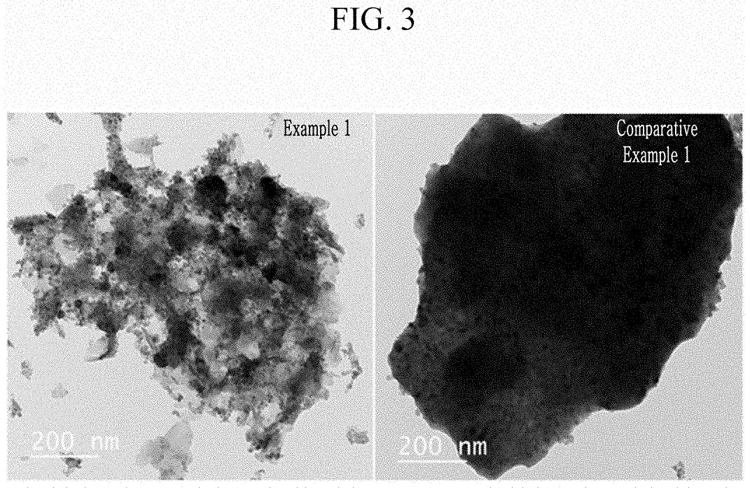

In order to examine physical/structural characteristics of the synthesized hybrid CZA/mesoFER catalyst, XRD, N₂-sorption, and TEM were performed, and the results are shown in FIGS. 1 to 3. In addition, BET specific surface area results measured by using N₂-sorption are shown in Table 4.

Through the XRD analysis, an XRD diffraction pattern unique to ferrierite and a diffraction pattern of Cu were simultaneously observed, and through the BET specific surface area results, an area change according to characteristics of support ferrierites was found. In addition, through the TEM image, structures of the support ferrierites, which affected the change of the specific surface area, were visually and clearly confirmed.

### [Experiment 4: Chemical Characterization Analysis of Hybrid CZA/mesoFER Catalyst]

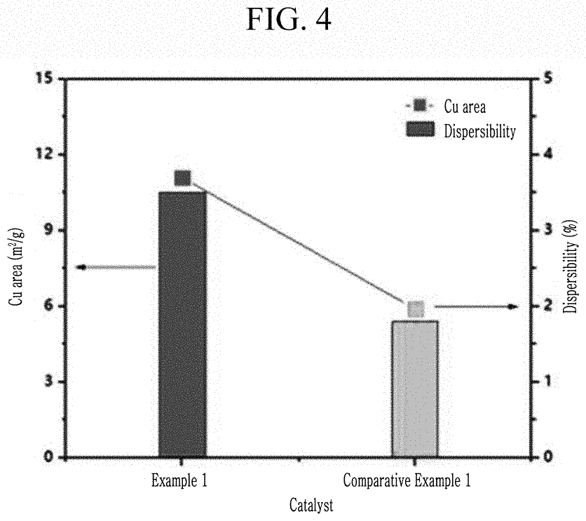

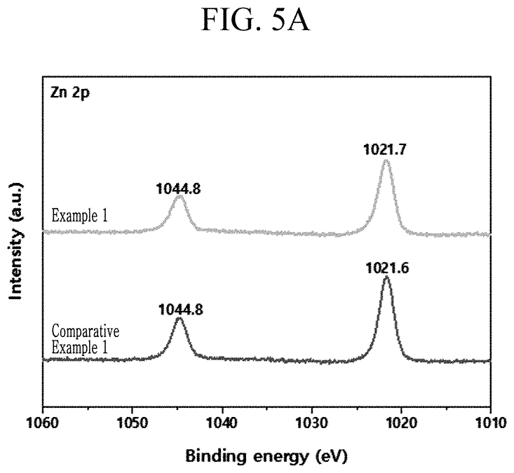

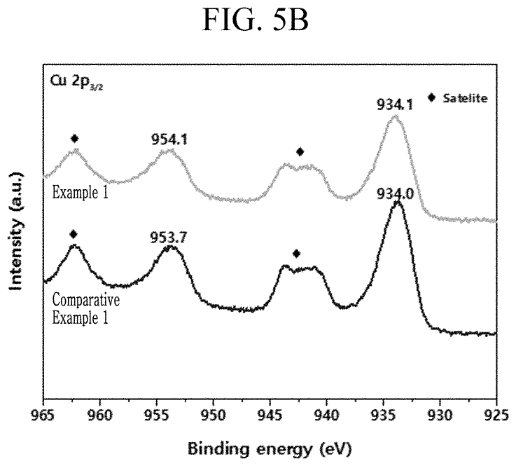

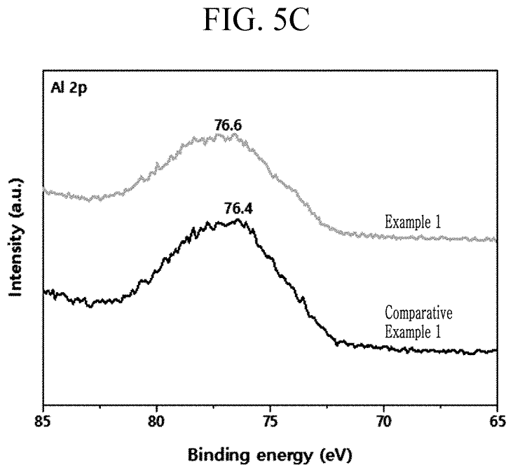

The synthesized hybrid CZA/mesoFER catalyst was analyzed with respect to chemical characteristics by performed N₂O-chemisorption, XPS, NH₃-TPD, and H₂-TPR, and the results are respectively shown in FIGS. 4 and 5 and Table 4.

After the N₂O-chemisorption analysis, the chemical adsorption patterns of N₂O and Cu were examined to quantitatively obtain an area and dispersibility of Cu on CZA/mesoFER. After performing XPS, a relative ratio of Cu, Zn, and Al metals on the CZA/mesoFER catalyst was measured. As for the NH₃-TPD analysis, after sufficiently adsorbing NH₃ in acid sites on CZA/mesoFER at 100° C., the temperature was increased up to 450° C., measuring an amount of the adsorbed NH₃. After measuring an area of a TPD pattern obtained after the analysis, this area was used to quantitatively obtain the acid sites on each CZA/mesoFER catalyst. In addition, an H₂-TPR analysis was performed to examine reducibility of CZA/mesoFER and stability of Cu species by using H₂. Herein, a TPR spectrum was obtained by increasing the temperature up to 400° C., while H₂/Ar=5/95 gas was continuously flowed, and then, sufficient oxidation was performed again by sufficiently using O₂/He=1/99 gas, and reduction also was performed again under the same condition. After the analysis, two consecutive reduction temperatures were checked to investigate how easily the Cu species were reduced in each support FER and whether or not reducibility thereof was maintained even after the oxidation.

TABLE 4

| | N₂-sorption | XPS | N₂O- | | |
|---|---|---|---|---|---|
| | [specific | [Cu/Al | chemi- | NH₃- | H₂-TPR |
| | surface area/ | ratio/ | sorption | TPD | [1^{st} reduction |
| | pore volume/ | Cu/Zn | [Cu area/ | [acid | temperature/ |
| | pore size] | ratio] | dis- | site] | 2^{nd} reduction |
| | (m²/g/cm³/ | (a.u/ | persibility] | (mmol/ | temperature] |
| Catalyst | g/nm) | a.u.) | (m²/g/%) | g) | (° C./° C.) |
| Example 1/ | 85.0/0.374/ | 2.34/ | 11.1/3.5 | 0.529 | 214/228 |
| CZA/ | 17.6 | 1.23 | | | |
| mesoFER | | | | | |
| Comparative | 82.3/0.269/ | 2.60/ | 5.9/1.8 | 0.555 | 214/230 |
| Example 1/ | 13.1 | 1.07 | | | |
| CZA/CFER | | | | | |

Referring to Table 4, even though metal precursors in an equal amount were coprecipitated in the same method depending on a type of support ferrierite, overall catalyst characteristics tended to be changed. The most obvious difference was found in distributions of Cu, a reaction point, that is, in mesoporous ferrierite having mesopores, a wider pore volume and also, higher Cu dispersibility due to the unique structure and thus a wider surface area of Cu per unit catalyst weight were observed than in commercially available ferrierite support. According to characteristics of support ferrierites, a catalyst exhibited an overall large specific surface area itself after supported, and even after the XPS analysis, a relatively large amount of Cu species was found. However, an amount of the acid sites in the ferrierite support where synthetic gas was finally converted into DME were larger than that in CZA/CFER co-precipitating Cu/ZnO/Al₂O₃ in commercially available FER. In addition, as a result of H₂-TPR, reduction degrees of Cu were similar in CZA/CFER and CZA/mesoFER. Accordingly, excellent reactivity of CZA/mesoFER was entirely attributed to dispersibility of Cu and an increase in a pore volume and a pore size due to a mesoporous structure, which is physical property of a catalyst.

Resultantly, Cu/ZnO/Al₂O₃ was co-precipitated on the mesoporous ferrierite support, developing excellent hybrid CZA/mesoFER and optimizing a reaction point and thereby, confirming high DME productivity.

While this disclosure has been described in connection with what is presently considered to be practical example embodiments, it is to be understood that the disclosure is not limited to the disclosed embodiments. On the contrary, it is intended to cover various modifications and equivalent arrangements included within the spirit and scope of the appended claims.

What is claimed is:

1. A method for manufacturing a catalyst for synthesizing dimethyl ether from synthetic gas, comprising:
preparing a mesoporous ferrierite zeolite (FER); and
co-precipitating a precursor of a mesoporous ferrierite zeolite and a Cu—Zn—Al-based oxide (CZA) to obtain a hybrid CZA/mesoFER catalyst.

2. The method of claim 1, wherein the preparing of the mesoporous ferrierite zeolite comprises:
preparing ferrierite;
silicon-leaching the ferrierite; and
performing hydrothermal synthesis of the precursor mixed solution.

3. The method of claim 2, wherein the preparing of ferrierite comprises:
adding a silica source, an alumina source, and a ferrierite seed to a basic aqueous solution to prepare a precursor mixed solution; and
synthesizing ferrierite by hydrothermal synthesis of the precursor mixed solution.

4. The method of claim 3, wherein the ferrierite seed is added in an amount of about 2 wt % to about 30 wt % based on the total weight of the prepared ferrierite.

5. The method of claim 3, wherein the hydrothermal synthesis of the precursor mixed solution is performed at about 120° C. to about 180° C. for about 96 hours to about 168 hours.

6. The method of claim 2, wherein the silicon-leaching of the ferrierite is performed by adding an organic template material and ferrierite to a basic aqueous solution and stirring at about 10° C. to about 80° C. for about 1 hour to about 15 hours.

7. The method of claim 6, wherein the organic template material is a linear organic compound having 15 to 30 carbons and at least one nitrogen.

8. The method of claim 6, wherein the organic template material comprises cetrimonium bromide (CTAB), sodium dodecyl sulfate, ammonium lauryl sulfate, or a combination thereof.

9. The method of claim 6, wherein the organic template material is added in an amount of about 10 to about 50 parts by weight based on 100 parts by weight of ferrierite.

10. The method of claim 2, wherein the hydrothermal synthesis of silicon-leached ferrierite is performed at about 120° C. to about 180° C. for about 48 hours to about 96 hours.

11. The method of claim 2, wherein the hydrothermal synthesis of silicon-leached ferrierite further comprises ion-exchanging a Na-form zeolite prepared by hydrothermal synthesis of silicon-leached ferrierite with a cation to prepare a NH₃-form zeolite.

12. The method of claim 11, wherein the hydrothermal synthesis of silicon-leached ferrierite further comprises calcining the ion-exchanged zeolite at about 450° C. to about 650° C. for about 3 hours to about 6 hours to convert the ion-exchanged zeolite into H-from zeolite.

13. The method of claim 1, wherein the co-precipitating comprises:
preparing a first solution including mesoporous ferrierite zeolite;
preparing a second solution including a copper precursor, a zinc precursor, and an aluminum precursor;
preparing a third solution including a basic precipitating agent; and
adding the second solution and the third solution to the first solution to perform co-precipitating.

14. The method of claim 13, wherein a mole ratio of Cu:Zn:Al in the second solution is (about 10 to about 5):(about 5 to about 1):1.

15. The method of claim 13, wherein
the copper precursor comprises an acetate, a hydroxide, a nitrate, or a combination thereof of copper,
the zinc precursor comprises an acetate, a hydroxide, a nitrate, or a combination thereof of zinc, and
the aluminum precursor comprises an acetate, a hydroxide, a nitrate, or a combination thereof of aluminum.

16. The method of claim 13, wherein the basic precipitating agent comprises sodium carbonate, potassium carbonate, ammonium carbonate, sodium hydrogen carbonate, or a combination thereof.

17. The method of claim 13, wherein in the co-precipitating process, the second solution and the third solution are dropped dropwise to the first solution to co-precipitate the precursor of the mesoporous ferrierite zeolite and the Cu—Zn—Al-based oxide (CZA).

18. The method of claim 13, wherein the co-precipitating is performed at a temperature of about 65° C. to about 75° C. and a pH of less than or equal to about 7.

19. The method of claim 13, wherein the co-precipitating further comprises growing a crystal of the prepared precipitate for about 1 hour to about 2 hours.

20. The method of claim 13, wherein the co-precipitating further comprises calcining the prepared precipitate at about 200° C. to about 600° C. for about 2 hours to about 6 hours.

21. A hybrid CZA/mesoFER catalyst, comprising
a mesoporous ferrierite zeolite, and
a Cu—Zn—Al-based oxide supported on the mesoporous ferrierite zeolite.

22. The hybrid CZA/mesoFER catalyst of claim 21, wherein the Cu—Zn—Al-based oxide comprises about 40 wt % to about 60 wt % of CuO, about 35 wt % to about 45 wt % of ZnO, and about 5 wt % to about 15 wt % of Al₂O₃ based on the total weight of the Cu—Zn—Al-based oxide.

23. The hybrid CZA/mesoFER catalyst of claim 21, wherein the hybrid CZA/mesoFER catalyst comprises about 0.1 part by weight to about 5 parts by weight of the Cu—Zn—Al-based oxide based on 1 part by weight of the mesoporous ferrierite zeolite.

24. The hybrid CZA/mesoFER catalyst of claim 21, wherein a Si/Al ratio of the mesoporous ferrierite zeolite is about 5 to about 30.

25. The hybrid CZA/mesoFER catalyst of claim 21, wherein the mesoporous ferrierite zeolite has mesopores having a size of about 10 nm to about 70 nm in an amount of about 80 volume % to about 30 volume %.

26. A method for synthesizing dimethyl ether includes selectively synthesizing dimethyl ether through a conversion reaction of synthetic gas using the hybrid CZA/mesoFER catalyst of claim 21.

27. The method of claim 26, wherein
in the method for synthesizing dimethyl ether,
the synthetic gas comprises hydrogen (H₂) and carbon monoxide (CO) in a mole ratio of about 1:2.5 to about 1:7.5, and
the synthetic gas comprises about 8 mol % to about 30 mol % of carbon monoxide based on the total amount of the synthetic gas.
